(19) United States
(12) Patent Application Publication
(10) Pub. No.: US 2010/0119653 A1
(43) Pub. Date: May 13, 2010

(54) NUTRITIONAL SUPPLEMENTS AND THEIR METHODS OF FORMULATION

(76) Inventor: Edward Hall, Sandy, UT (US)

Correspondence Address: THORPE NORTH & WESTERN, LLP., P.O. Box 1219, SANDY, UT, 84091-1219, US

(21) Appl. No.: 12/617,627

(22) Filed: Nov. 12, 2009

Related U.S. Application Data
(60) Provisional application No. 61/114,281, filed on Nov. 13, 2008.

(51) Int. Cl. A23L 1/28; C12Q 1/68; A23L 1/212

(52) U.S. Cl. 426/62; 435/6; 426/615

(57) ABSTRACT

A method of formulating a nutritional supplement and related nutritional supplements are taught. The method involves the steps of identifying the target population, identifying the genetic haplogroups present in the target population, and then identifying the ancestral migratory pathways for each haplogroup present in the target population. After identification of the migratory pathways for each haplogroup present in the target population, the major geographic regions along each migratory pathway where the haplogroups spent significant time are identified. Based on the major geographic regions identified, natural whole foods indigenously present in major geographic regions are then identified and incorporated into a nutritional supplement.

This application claims the benefit of U.S. Provisional Application No. 61/114,281, filed Nov. 13, 2008.

### FIELD OF THE INVENTION

The present disclosure relates generally to nutritional supplements and related methods of formulation. More particularly, the present disclosure relates to the use of genetics in the formulation of nutritional supplements.

### BACKGROUND

Genetics and nutrition play critical roles in the health of people throughout the world. Proper nutrition can prevent dietary deficiencies and can aid in protecting against the development of many diseases. Similarly, an individual's genetic makeup has been linked to the individual's susceptibility to physiological stress and disease. Other non-genetic factors are also believed to have an effect on overall health and gene expression. These non-genetic factors are often referred to as epigenetic factors and can have an impact in overall gene expression without changing the underlying DNA sequence of a gene, thereby having an impact on the overall well-being of an individual. Although significant work has been done to identify genes which can render individuals susceptible to disease and many nutrients and plants have been identified as being potentially beneficial to human health, a need still remains in correlating genetic makeup with nutritional intake in order to positively and appropriately impact epigenetic factors and, thereby, overall gene expression.

### SUMMARY

The present disclosure is drawn to a method of formulating a nutritional supplement and related nutritional supplements. In particular, a method of present disclosure provides for a method of formulating a nutritional supplement for a target population. The method involves the steps of identifying the target population, identifying the major genetic haplogroups present in the target population, and then identifying the ancestral migratory pathways for each of the major haplogroups present in the target population. After identification of the migratory pathways for each major haplogroup present in the target population, the major geographic regions along each migratory pathway where the major haplogroups spent significant time are identified. Based on the major geographic regions identified, natural whole foods indigenous to the major geographic regions are then identified, selected, and incorporated into a nutritional supplement.

In another embodiment, a nutritional supplement is provided. The nutritional supplement can include a collection of natural whole food components including a first group of ingredients, a second group of ingredients, a third group of ingredients, and a fourth group of ingredients. The first group of ingredients can comprises about 55 wt % to about 65 wt % of the collection of natural whole food components and can include at least three first group ingredients selected from polygonum cuspidatum, amla, turmeric, curcumin, astragalus, fo-ti, green tea, ginkgo biloba, broccoli, pagoda tree flower bud, goji berry, mangosteen, noni, asian ginseng, eleuthero siberian ginseng, ashwaghanda, rhodiola, reishi mushroom, cordyceps, schizandra berry, chlorella, spirulina, barley grass, wheat grass, ginger, boswellia, holy basil, bitter orange, sweet orange, lemon, luohanguo, guggul, cinnamon, maitake, lychee, arjuna, white tea, hemp, epimedium sagittatum, dandelion, chamomile, flax seed, cranberry, spinach, carrot, fermented yeast/inactive nutritional yeast, fulvic acid/humic acid plant based trace minerals, and black pepper. The second group of ingredients can comprise about 25 wt % to 35 wt % of the collection of natural whole food components and can include at least two second group ingredients selected from olive leaf, dandelion, grapes, grapeseed, tribulus, marigold, carrot, flax seed, pomegranate, black currant, elderberry, bilberry, hawthorne berry, kale, brussels sprouts, spinach, fulvic acid/humic acid plant based trace minerals, honey, milk thistle, fenugreek, fig, ferula hurmonis zallouh, date, borage, parsley, pistachio, walnut, lavandula officinalis, lemon balm, saffron, butcher's broom, watercress, rose hips, cranberry, barley grass, wheat grass, and broccoli sprouts. The third group of ingredients can comprise about 5 wt % to 15 wt % of the collection of natural whole food components and can include at least one third group ingredient selected from devils claw, marulu tree fruit, yohimbe, roobios, kigelia, hoodia, spirulina, chamomile, black seed, and aloe vera, and wherein the third group of ingredients comprises. The fourth group of ingredients can comprise about 5 wt % to about 15 wt % of the collection of natural whole food components and can include at least one fourth group ingredient selected from noni, coconut, marine phytoplankton, marine algae, banaba, tongkat ali, kakadu plum, kava kava, bitter orange, mangosteen, spirulina, laver/nori, bladderwrack, and kelp.

There has thus been outlined, rather broadly, the more important features of the disclosure so that the detailed description thereof that follows may be better understood, and so that the present contribution to the art may be better appreciated. Other features of the present disclosure will become clearer from the following detailed description, taken with the accompanying drawings and claims, or may be learned by the practice of the invention.

### BRIEF DESCRIPTION OF THE DRAWINGS

FIG. 1 shows a map of the Western Hemisphere which shows major migratory pathways of ancient man, and in particular major migratory pathways for specific haplogroups.

FIG. 2 shows a magnified region of FIG. 1 including all of South and Central America and some major migratory pathways of ancient man to these regions.

FIG. 3 shows a magnified region of FIG. 1 including the United States and Canada and some major migratory pathways of ancient man to these regions.

FIG. 4 shows a magnified region of FIG. 1 including Alaska and the Bering Strait and some of the major migratory pathways of ancient man to and through these regions.

FIG. 5 shows a map of the Eastern Hemisphere which shows major migratory pathways of ancient man, and in particular major migratory pathways for specific haplogroups.

FIG. 6 shows a magnified region of FIG. 5 including Australia and the majority of East Asia and some of the major migratory pathways of ancient man to and through these regions.

FIG. 7 shows a magnified region of FIG. 5 including portions of East and Central Asia and some of the major migratory pathways of ancient man to and through these regions.

FIG. 8 shows a magnified region of FIG. 5 including most of Europe and Africa and some of the major migratory pathways of ancient many to and through these regions.

FIG. 9 shows a magnified region of FIG. 5 including portions of the Middle East and Europe and some of the major migratory pathways of ancient man to and through these regions.

### DETAILED DESCRIPTION

Before the present invention is disclosed and described, it is to be understood that this disclosure is not limited to the particular structures, process steps, or materials disclosed herein, but is extended to equivalents thereof as would be recognized by those ordinarily skilled in the relevant arts. It should also be understood that terminology employed herein is used for the purpose of describing particular embodiments only and is not intended to be limiting.

In describing and claiming the present invention, the following terminology will be used in accordance with the definitions set forth below.

It is noted that, as used herein, the singular forms of “a,” “an,” and “the” include plural referents unless the context clearly dictates otherwise. Thus, for example, reference to “haplogroup” includes one or more of such haplogroups, and reference to “the migratory pathway” includes references to one or more migratory pathways.

### DEFINITIONS

As used herein, the term “target population” refers to a group of human subjects. The size of the target population can vary from hundreds to millions depending on the desired scope of the nutritional supplement being formulated. The target population can be identified by genetic traits, geographic location, or other qualifier. Examples of target populations include peoples of North America, peoples of Japan, peoples of Central Asia, or other groups of peoples from other large geographic areas.

As used herein, the term “haplogroup” refers to a group of human subjects having similar genetic haplotypes that share a common ancestor with a single nucleotide polymorphism (SNP) mutation. Haplogroups are assigned letters of the alphabet, and refinements consist of additional number and letter combinations. Generally, haplogroups can be Y-chromosome haplogroups or mitochondrial DNA haplogroups. Y-chromosome and mitochondrial DNA haplogroups have different haplogroup designations. Haplogroups are well known in the art and pertain to deep ancestral origins dating back thousands of years. The term “major haplogroups” refers to haplogroups present in at least 5% of a designated target population. Accordingly, the term “major haplogroup” is tied to a particular target population as a major haplogroup in one target population may not be a major haplogroup for another target population.

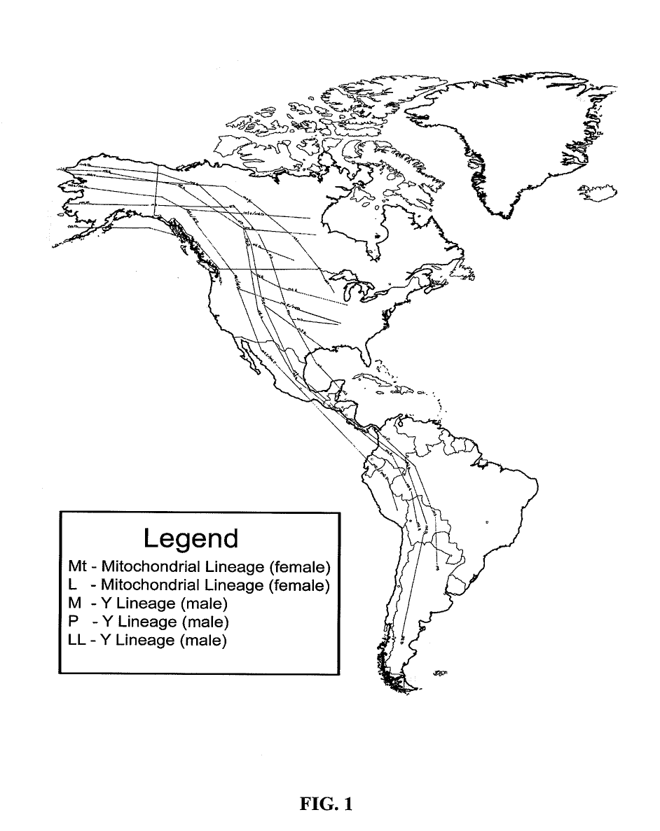

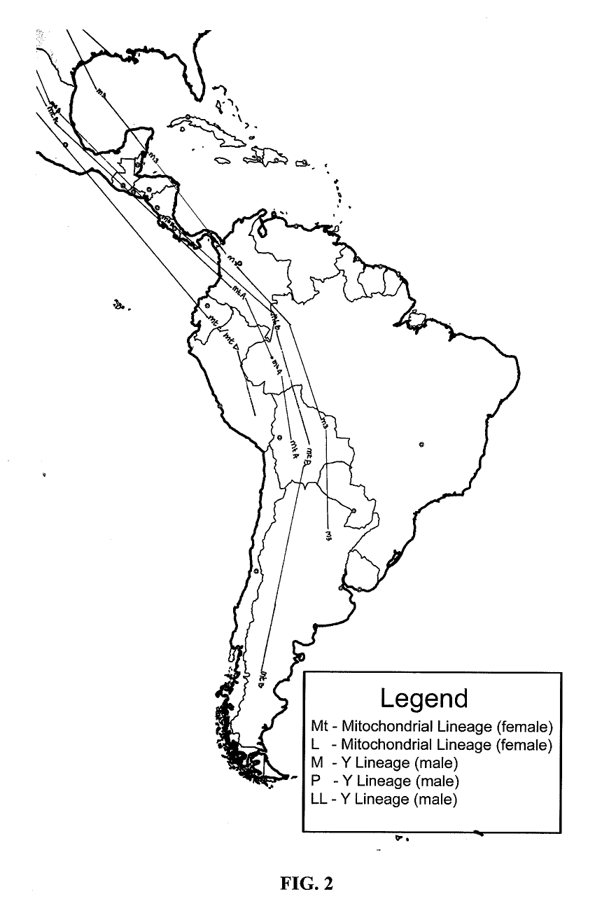

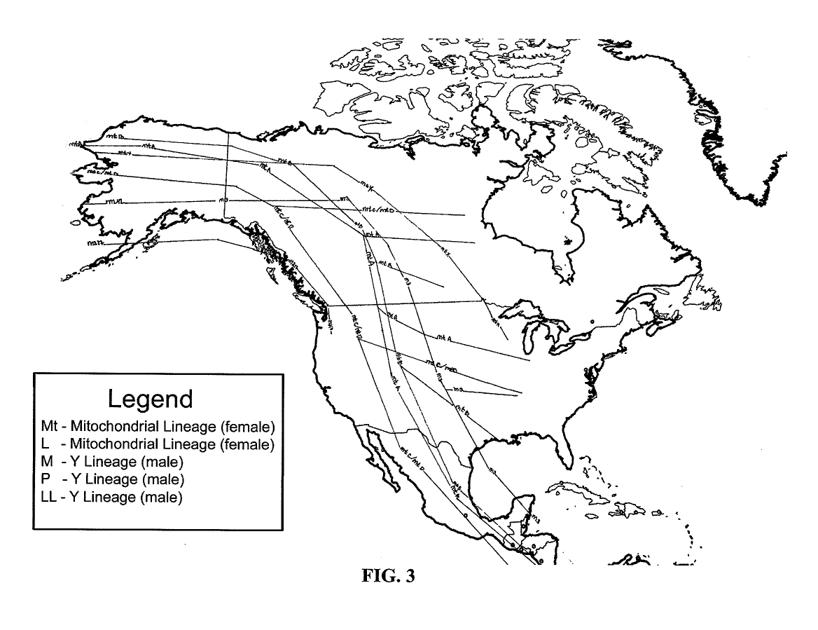

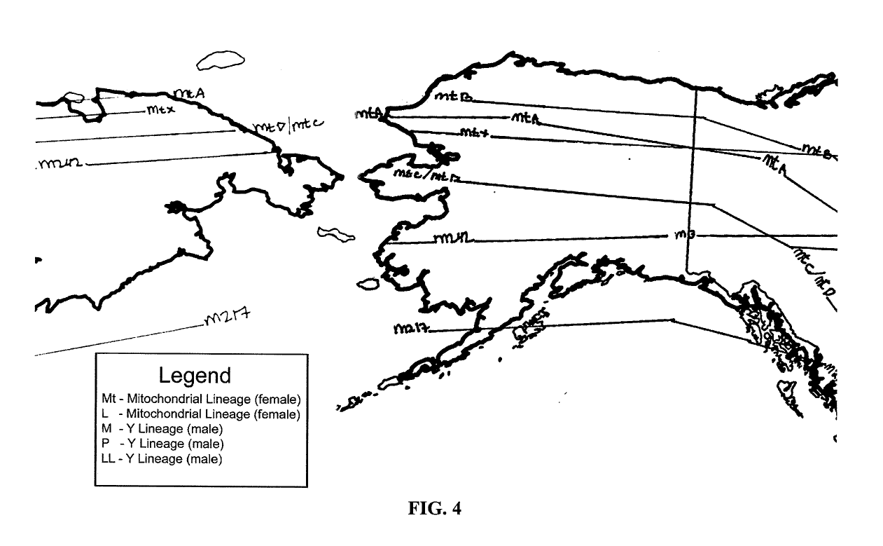

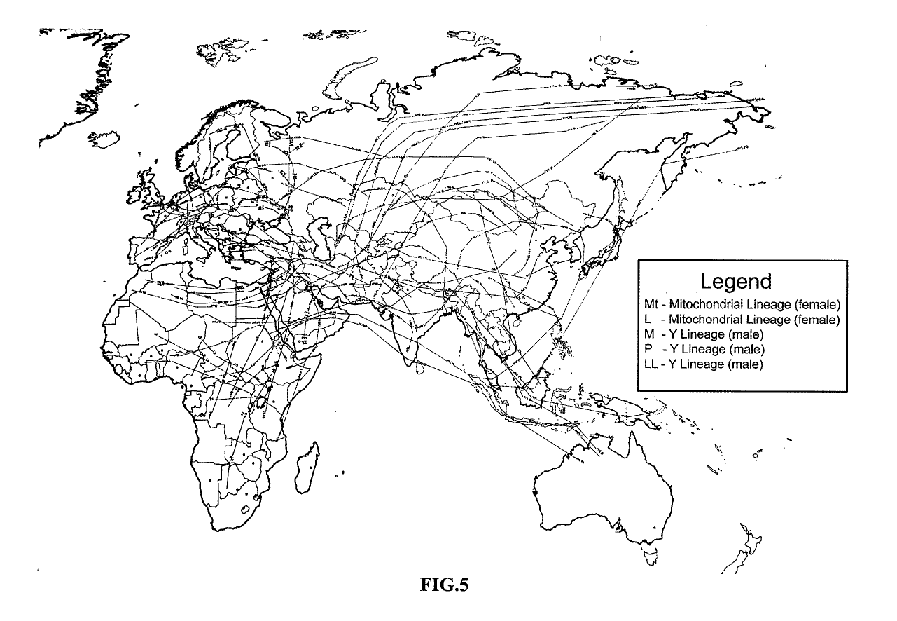

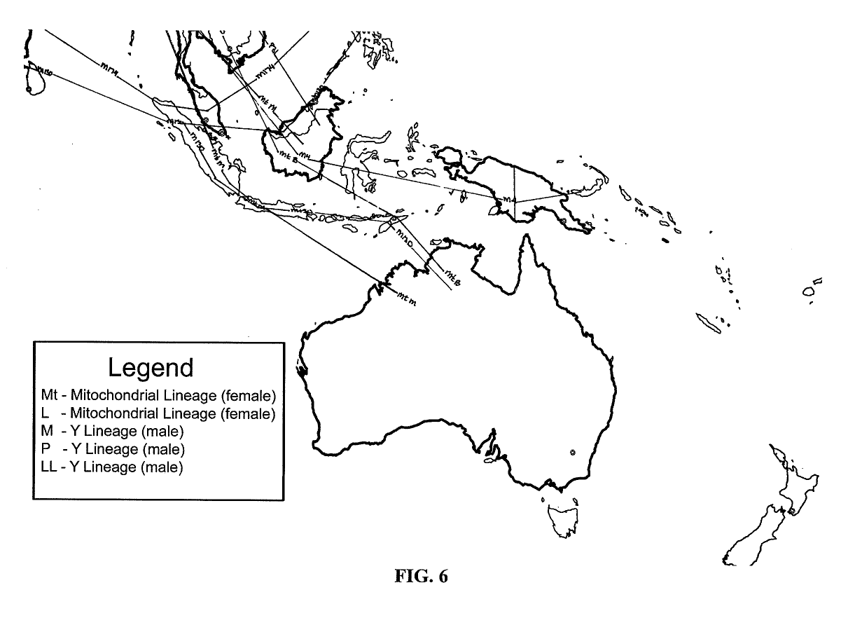

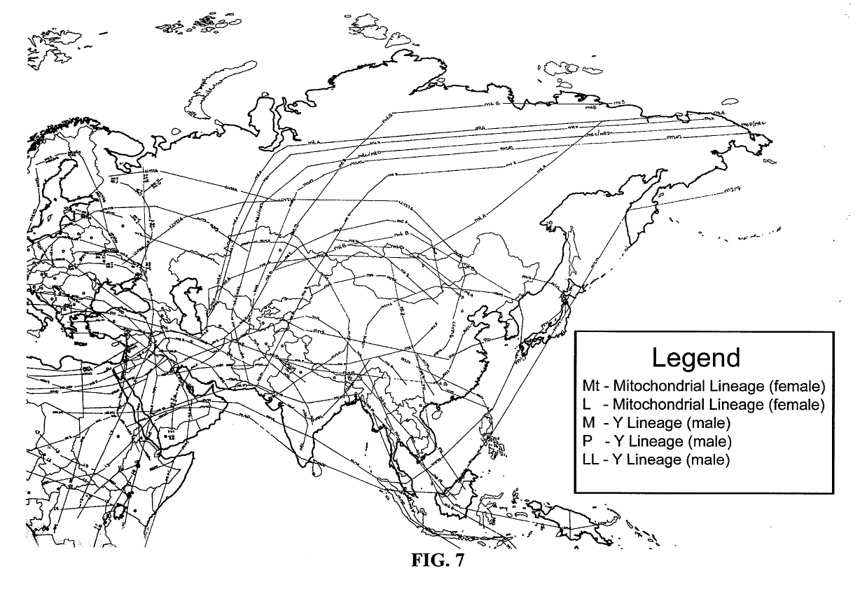

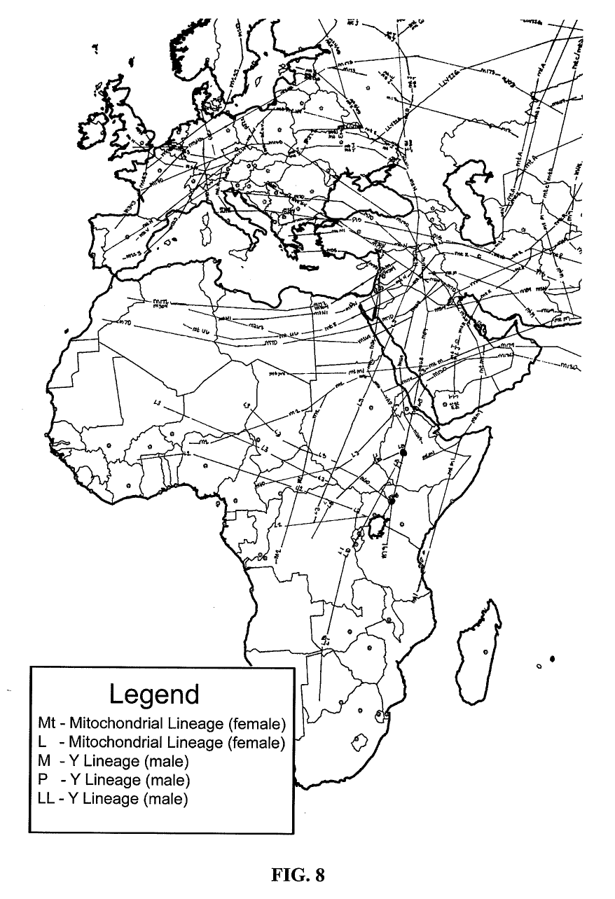

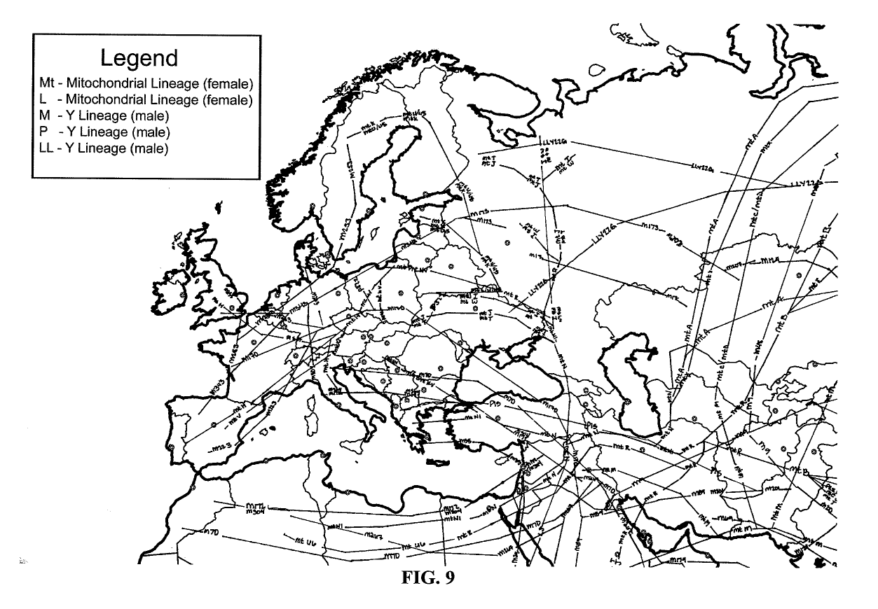

The phrase “migratory pathways” refers to the major migration routes of ancient man. Such pathways generally correlate the origin of specific haplogroups and their geographic spread and branching over time. In other words, the term “migratory pathways” does not necessarily provide the precise route taken by a specific group of people from one geographic point to another but rather the generalized spread of a haplogroup from one geographic region to another over time. Some examples of migratory pathways for various haplogroups are shown in FIGS. 1-9. It is noted that in FIGS. 1-9, locations of angles, turns, or branches in the migratory pathways can be indicative of regions where the haplogroup spent a significant period of time.

The phrase “major geographic regions” refers to geographic locations or regions where haplogroups are known to have spent significant time. As used herein the term “significant period of time” is at least about 5,000 years. In one embodiment, the significant period of time occurs during the time period of 50,000 years ago to the last glacial maximum (about 20,000) years. In another embodiment, the significant period of time occurs during the time period of pre-50,000 years (earliest known man) to about 50,000 years ago. In a further embodiment, the significant period of time occurs during the time period of about 20,000 years ago to the present. A formulation can be formulated based on significant periods of time from any of the above time periods. Non-limiting examples of major geographic regions include Africa, the Mediterranean (includes coastal regions surrounding the Mediterranean sea), Middle East (including the countries of Turkey, Syria, Israel, Saudi Arabia, Iran, Iraq, portions of Afghanistan, Pakistan, and Turkmenistan), Europe (divided from Asia by the Ural mountains, the Ural River, the Caspian Sea and the Caucasus Mountains), Central Asia (including from the Caspian Sea in the west to central China in the east and from southern Russia in the north to northern Pakistan in the south—including the at least portions of the present countries of Kazakhstan, Afghanistan, Mongolia), India, Himalaya (including the mountainous region between India, Pakistan, Nepal, and Tibet), Siberia (including northern Asia, northern China, northern Mongolia, Russia, and north-central Kazakhstan), East Asia (includes the eastern portion of China, Korea, Taiwan, eastern Mongolia, Japan), Southeast Asia (includes Thailand, Vietnam, Malaysia, Philippines, Taiwan, and portions of Indonesia), Oceanic (includes islands and oceans off southeast Asia, Indonesia, Oceania, and the islands and oceans off Europe), and combinations thereof.

The phrase “natural whole foods” are those whole plants or plant parts used for foods or herbs that are unprocessed and unrefined, or processed and refined as little as possible before being consumed. They typically do not contain added ingredients, such as sugar, salt, or fat. The term natural whole foods can include whole plant powders and extracts made from the natural whole plant.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 wt % to about 5 wt %” should be interpreted to include not only the explicitly recited values of about 1 wt % to about 5 wt %, but also include individual values and sub-ranges within the indicated range. Thus, included in this numerical range are individual values such as 2, 3.5, and 4 and sub-ranges such as from 1-3, from 2-4, and from 3-5, etc. This same principle applies to ranges reciting only one numerical value. Furthermore, such an interpretation should apply regardless of the breadth of the range or the characteristics being described.

The present disclosure is drawn to a method of formulating a nutritional supplement and related nutritional supplements. In particular, a method herein provides for a method of formulating a nutritional supplement for a target population. The method involves the steps of identifying the target population, identifying the major genetic haplogroups present in the target population, and then identifying the ancestral migratory pathways for each major haplogroup present in the target population. After identification of the migratory pathways for each major haplogroup present in the target population, the major geographic regions along each migratory pathway where the major haplogroups spent significant time are identified. Based on the major geographic regions identified, natural whole foods indigenously present in major geographic regions are then identified and incorporated into a nutritional supplement.

The methods of the present disclosure can be applied to formulate a nutritional supplement for any target population. Although the target population can be of any size, for commercial feasibility it can be beneficial to have a target population which has a relatively large size, e.g. several hundred thousand to several hundred million.

Once a target population is identified, the major haplogroups present in the target population can be identified. Identification of the major haplogroups present in a target population can be done either through analysis of each member of the target population or through statistical analysis based on a representative group of the target population. As target populations can be large, the identification of representative major haplogroups can be preferably done through statistical analysis based on a representative group of the target population. When target populations are very large they have the propensity to include nearly all haplogroups. In such situations, the formulating method may be revised so that only the major haplogroups or most prevalent haplogroups of a target population are included in the method, thereby formulating a nutritional supplement which is effective for the vast majority of the target population, but not the entire target population. Any genetic testing method known in the art can be used to identify the haplogroups present in a particular subject.

In one embodiment, the target population can be the people of North America. The North American target population is an example of a target population which is diverse and has a large number of haplogroups present in the population. In formulating a nutritional supplement for a North American target population it may be advantageous to focus on the haplogroups which are most prevalent, e.g. major haplogroups, in order to provide a formulation which will be effective for the majority of the population, but not necessarily all members of the target population. In another embodiment, the target population could be the people of Eastern Asia, including Japan and/or Korea. In yet another embodiment, the target population could be the people of

Central Asia including the peoples of Pakistan, northern India, Central China, and southern Russia.

The number of genetic haplogroups present in a target population can vary by target population. Factors which can influence the number of haplogroups in the target population include the size of the target population, the size of the area covered by the target population, and the geography of the target population. There are a large number of haplogroups which are well known to those of ordinary skill in the art and discovery of new haplogroups is ongoing. Haplogroups are either mitochondrial (mtDNA) or Y-chromosome DNA (Y-DNA) haplogroups. Non-limiting examples of mitochondrial haplogroups include A, B, C, D, F, H, HV1, HV, I, J, K, L0, L1, L2, L3, M1, M, N1, N, Pre HV, R, T, U5, U6, U, V, W, X, and Z. Non-limiting examples of Y-DNA haplogroups include M119-01a, M91-A, M60-B, M217-C3, M130-C, M15-D1, P37.1-D2, M174-D, M2-E3a, M35-E3b, M96-E, M89-F, P15-G2, M201-G, M52-H1, M69-H, M253-I1a, P37.2-I1b, M223-I1c, M170-I, M267-J1, M172-J2, M304-J, M70-K2, M9-K, M9-K*, M20-L, LLY22-N, M168, M4-M, P31-O2, M122-O3, M175-O, M45-P, M3-Q3, M242-Q, M173-R1, M17-R1a1, M343-R1b, M124-R2, M207-R, and YAP.

Generally speaking, a target population can include any combination of the above mitochondrial and/or Y-DNA haplogroups. In one embodiment, a target population can include the mitochondrial haplogroups A, B, C, D, F, H, HV1, HV, I, J, K, L0, L1, L2, L3, M1, M, N1, N, R, T, U5, U6, U, V, W, X, Z. and the Y-DNA haplogroups M119, M91, M60, M217, M130, M15, P37.1, M174, M2, M35, M96, M89, P15, M201, M52, M69, M253, P37.2, M223, M170, M267, M172, M304, M70, M9, M20, LLY22, M168, M4, P31, M122, M175, M45, M3, M242, M173, M17, M343, M124, and M207.

After identification of the haplogroups, the ancestral migratory pathways for each of these haplogroups can be identified. The migratory pathways for most of the known haplogroups are known or can be readily determined by those of ordinary skill in the art. Once identified, the migratory pathways can be used to determine the major geographic regions where the haplogroups spent significant time. Non-limiting examples of such migratory pathways and the associated geographic regions for various haplogroups are shown the National Geographic website “The Genographic Project” which can be found at the following web address: https://www3.nationalgeographic.com/genographic (accessed October 2008).

For the purposes of the present disclosure, the major geographic regions are as follows: Africa; Mediterranean; Middle East; Europe—including northern, southern, eastern and western Europe; Central Asia—including from the Caspian Sea in the west to central China in the East and from southern Russia in the north to northern Pakistan and northern India in the south; India; Siberia; Southeast Asia—including eastern China, Thailand, Cambodia, Laos, and Vietnam; Eastern Asia—including Korea and Japan; Oceania—including islands and oceans off southeast Asia, Indonesia, and the islands and oceans off Europe). It is important to note that each haplogroup may have multiple major geographic regions where it spent significant time regions along its migratory pathway.

Once the major geographic regions where the haplogroups have spent significant time are identified, the next step is to identify and select natural whole foods which are indigenous to at least one of the major geographic regions (some of the natural whole foods may be indigenous to multiple major geographic regions). Identification and selection of foods can be accomplished by evaluating historical and current food habits of native peoples in the region. It is worth noting that current food habits may not always mimic or resemble the historical food habits of a region. Identification of foods indigenous to a particular region is within the knowledge of one of ordinary skill in the art. In one embodiment, the natural whole foods incorporated into the nutritional supplement can be grown in the major geographic region to which they are indigenous. Sourcing the natural whole foods from the regions where they are indigenous can be desirable because the nutritional, medicinal, or therapeutic effect of a natural whole food can be altered when grown in a region with different soil or climate makeup.

Non-limiting examples of natural whole foods include polygonum cuspidatum, amla, turmeric, curcumin, astragalus, fo-ti, green tea, ginko biloba, broccoli, pagoda tree flower bud, goji berry, mangosteen, noni, asian ginseng, eleuthero siberian ginseng, ashwaghanda, rhodiola, reishi mushroom, cordyceps, schizandra berry, chlorella, spirulina, barley grass, wheat grass, ginger, boswellia, holy basil, bitter orange, sweet orange, lemon, luohanguo, guggul, cinnamon, maitake, lychee, arjuna, white tea, hemp, epimedium sagittatum, dandelion, chamomile, flax seed, black pepper, olive leaf, dandelion, grapes, grapeseed, black seed, tribulus, marigold, carrot, flax seed, pomegranate, black currant, elderberry, bilberry, hawthorne berry, kale, brussels sprouts, spinach, fulvic acid/humic acid plant based trace minerals, honey, milk thistle, fenugreek, fig, ferula hurmonis zallouh, date, borage, parsley, pistachio, walnut, lavandula officinalis, lemon balm, saffron, butcher's broom, watercress, rose hips, laver/nori bladderwrack, and broccoli sprouts, and devils claw, marulu tree fruit, yohimbe, roobios, kigelia, hoodia, aloe vera, noni, coconut, marine phytoplankton, marine algae, banaba, tongkat ali, kakadu plum, kava kava, kelp, and combinations thereof.

In one embodiment of the present disclosure, the natural whole foods can be selected from the group consisting of polygonum cuspidatum, amla, olive leaf, turmeric, curcumin, astragalus, fo-ti, green tea, ginkgo biloba, broccoli, pagoda tree flower bud, goji berry, mangosteen, noni, asian ginseng, eleuthero siberian ginseng, ashwaghanda, rhodiola, reishi mushroom, cordyceps, schizandra berry, chlorella, spirulina, barley grass, wheat grass, ginger, boswellia, black pepper, dandelion, grapes, grapeseed, black seed, tribulus, marigold, carrot, flax seed, pomegranate, black currant, elderberry, bilberry, hawthorne berry, holy basil, kale, brussels sprouts, spinach, broccoli sprouts, devils claw, aloe vera, chamomile, coconut, marine phytoplankton, kelp, bitter orange, sweet orange, lemon, fulvic acid/humic acid plant based trace minerals, luohanguo, honey, guggul, marine algae, cinnamon, milk thistle, fenugreek, fig, ferula hurmonis zallouh, banaba, tongkat ali, kakadu plum, date, borage, parsley, pistachio, walnut, lavandula officinalis, lemon balm, saffron, butcher's broom, watercress, rose hips, laver/nori, bladderwrack, maitake, marulu tree fruit, yohimbe, roobios, lychee, arjuna, kigelia, white tea, hoodia, hemp, kava kava, epimedium sagittatum, and combinations thereof.

Once identified, the selection and ultimate incorporation of the natural whole foods in a nutritional supplement can be done based on a variety of factors. One factor that can be used is the known or postulated scientific, medicinal, or therapeutic value of the natural whole food. For example, some foods, e.g. rice, are and have been used heavily in the diets of people in many areas of the world. However, aside from the general nutritional value of the rice, there is very little evidence that the consumption of rice provides any particular health or medicinal benefit. Accordingly, a natural whole food which is known to be indigenous to a particular region and is also known or postulated to have a particular medicinal or health benefit might be preferable to the rice.

Another factor relating to the selection and incorporation of the natural whole foods can relate to the relative ratio or percentages of haplogroups present in the target population and/or the relative duration of time the haplogroups spent in each major geographic area. For example, if 60% of the haplogroups in the target population spent significant time in Europe, 20% Africa, 20% Central Asia, and 10% Oceanic, then the nutritional supplement could be formulated to include natural whole foods from those regions and which natural whole foods could be included in the nutritional supplement in amounts that are somewhat proportional to the presence of the haplogroups in the target population. Known historical and or cultural use can also be a factor in selecting the natural whole foods which are included in the nutritional supplement.

Depending on the target population and the desired nature of the nutritional supplement, the selection and incorporation of the natural whole foods can also be influenced by desires to create a broad nutritive profile. For example, it may be desirable to have a balance of various types of foods such as different representative food/herbs from groups such as berries, fruits, grasses, greens, mushrooms, leaves, seeds, tubers, flowers, roots, seaweed, micronutrients (e.g. algae). In another embodiment, the selection of natural whole foods may be influence by the nutritional profile of the foods such as the presence and quantity of vitamins, minerals, amino acids, fiber, fatty acids, trace elements, antioxidants, polysaccharides, carotenoids, bioflavonoids, phytonutrients, and the like.

The nutritional supplements formulated by the methods of the present disclosure can be manufactured into various forms. Non-limiting examples of forms into which the nutritional supplement can be manufacture include powders, gels, liquids, other solid forms, and combinations thereof. The nutritional supplement can include other components such as excipients, flavorants, carriers, and combinations thereof. Any excipient, carrier or flavorant known in the art can be used. In one embodiment, the flavorant can be an ingredient which is not an ingredient of the collection of natural whole foods components. In another embodiment, the natural whole food component can be a natural whole food component. In yet a further embodiment, the nutritional supplement includes a gelling agent. Any gelling agent known in the art can be used.

One nutritional supplement formulated using the method of the present disclosure includes a collection of natural whole food components. The natural whole food components include a first group of ingredients, a second group of ingredients, a third group of ingredients, and a fourth group of ingredients. The first group of ingredients can include the natural whole foods polygonum cuspidatum, amla, turmeric, curcumin, astragalus, fo-ti, green tea, ginkgo biloba, broccoli, pagoda tree flower bud, goji berry, mangosteen, noni, asian ginseng, eleuthero siberian ginseng, ashwaghanda, rhodiola, reishi mushroom, cordyceps, schizandra berry, chlorella, spirulina, barley grass, wheat grass, ginger, boswellia, holy basil, bitter orange, sweet orange, lemon, luohanguo, guggul, cinnamon, maitake, lychee, arjuna, white tea, hemp, epimedium sagittatum, dandelion, chamomile, flax seed, fulvic acid/humic acid plant based trace minerals, fermented yeast/inactive nutritional yeast, black seed, and black pepper. The collection of natural whole food component includes at least three first group ingredients and the first group ingredients can make up about 55 wt % to about 65 wt % natural whole food components. In another embodiment the first group ingredient can make up about 57 wt % to about 63 wt % of the natural whole food components. In yet another embodiment the first group ingredients can make up about 59 wt % to about 61 wt % of the natural whole food components.

The second group of ingredients can include the natural whole foods olive leaf, chamomile, dandelion, grapes, grapeseed, tribulus, marigold, carrot, flax seed, pomegranate, black currant, elderberry, bilberry, hawthorne berry, kale, brussels sprouts, spinach, fulvic acid/humic acid plant based trace minerals, honey, milk thistle, fenugreek, fig, ferula hurmonis zallouh, date, borage, parsley, pistachio, walnut, lavandula officinalis, lemon balm, saffron, butcher's broom, watercress, rose hips, and, and broccoli sprouts. The collection of natural whole food component includes at least two second group ingredients and the second group ingredients can make up about 25 wt % to 35 wt % of the collection of natural whole food components. In another embodiment the second group ingredients can make up about 27 wt % to about 33 wt % of the natural whole food components. In yet another embodiment the second group ingredients can make up about 29 wt % to about 61 wt % of the natural whole food components.

The third group of ingredients can includes devils claw, marulu tree fruit, yohimbe, roobios, kigelia, hoodia, chamomile, black seed, and aloe vera. The collection of natural whole food component includes at least one third group ingredient and the third group ingredients can make up about 5 wt % to 15 wt % of the collection of natural whole food components. In another embodiment the third group ingredients can make up about 7 wt % to about 13 wt % of the natural whole food components. In yet another embodiment the third group ingredients can make up about 9 wt % to about 11 wt % of the natural whole food components. The fourth group of ingredients includes noni, coconut, marine phytoplankton, marine algae, banaba, tongkat ali, kakadu plum, kava kava, bitter orance, laver/nori, bladderwrack, and kelp. The collection of natural whole food component includes at least one fourth group ingredient and the fourth group ingredients can make up about 5 wt % to about 15 wt % of the collection of natural whole food components. In another embodiment the fourth group ingredient can make up about 7 wt % to about 13 wt % of the natural whole food components. In yet another embodiment the fourth group ingredient can make up about 9 wt % to about 11 wt % of the natural whole food components.

In one embodiment of the above described nutritional supplement, the collection of natural whole food components can include at least 40 different ingredients. In another embodiment, the collection of natural whole food components can include at least 45 different ingredients. In another embodiment, the first group of ingredients can include at least seven first group ingredients. In yet another embodiment, the second group of ingredients can include at least five second group ingredients. In a further embodiment, the third group of ingredients can include at least two third group ingredients. In yet a further embodiment, the fourth group of ingredients can include at least two fourth group ingredients.

### Examples

### Example 1

### Nutritional Supplement Formulation for North American Target Population

A nutritional supplement is formulated according to the method of the present disclosure. The target population is the people of North America, in particular the people of the United States and Canada. The haplogroups for the target population people are identified by tracing the molecular genealogy of a representative North American population. The most prevalent haplogroups identified in the North American population are mtDNA A, B, C, D, F, H, HV1, HV, I, J, K, L0, L1, L2, L3, M1, M, N1, N, R, T, U5, U6, U, V, W, X, Z. Y-DNA M119, M91, M217, M130, M15, P37.1, M174, M2, M35, M96, M89, P15, M201, M52, M69, M253, P37.2, M223, M170, M267, M172, M304, M170, M9, M20, LLY22, M4, P31, M122, M175, M45, M3, M242, M173, M17, M343, M124, M207. The genealogical timelines of the haplogroups are traced and the relevant ancestral migratory pathways for each of the groups are identified. Based on the migratory pathways, the major geographic regions where the haplogroups spent significant time are identified. The major geographic regions which are identified are Africa, Europe, Mediterranean, Middle East, India, Siberia, Central Asia, Oceania, South East Asia, and East Asia.

Based on the proportion of the haplogroups in the target population and the time spent by the haplogroups in the major geographic regions the contribution of the identified major geographic regions are estimated to be: Asia (Central Asia, East Asia, India, Siberia-taken together as Region 1): about 55-60%; Europe (Europe Mediterranean and Middle East—taken together as Region 2): about 30-35%; Africa (Region 3): about 10-15%; and Oceanic (Southeast Asia, Oceania, and Ocean waterways taken together as Region 4): about 10-15%. Natural whole foods from each of the above major geographic regions are identified. Approximately 45-65 of the identified whole foods are selected for inclusion and selected based on their origin, their known cultural and historic use, and their known or postulated medicinal or therapeutic value.

The following natural whole foods are identified and selected for inclusion. The natural whole foods are listed by the geographic region to which they pertain. Region 1: polygonum cuspidatum, amla, olive leaf, turmeric, fermented yeast/inactive nutritional yeast, astragalus, fo-ti, green tea, ginkgo biloba, broccoli, pagoda tree flower bud, goji berry, mangosteen, noni, asian ginseng, eleuthero siberian ginseng, ashwaghanda, rhodiola, reishi mushroom, cordyceps, schizandra berry, chlorella, spirulina, barley grass, wheat grass, ginger, boswellia, watercress, bitter orange, holy basil, luahanguo, cinnamon, fulvic acid/humic acid plant based trace minerals, and black pepper. Region 2: olive leaf, dandelion, grapes, grapeseed, black seed, tribulus, marigold, carrot, flax seed, pomegranate, black currant, elderberry, bilberry, hawthorne berry, holy basil, kale, brussels sprouts, spinach, milk thistle, cranberry, fulvic acid/humic acid plant based trace minerals, and broccoli sprouts. Region 3: devils claw, aloe vera, black seed, and chamomile. Region 4: coconut, marine phytoplankton, mangosteen, and kelp.

The amount (wt %) of each of the selected ingredients included in the formulation is approximated relative to the proportion of the haplogroups in the target population and the time spent by the haplogroups in the major geographic regions. A powdered nutritional supplement is made by blending each of the selected natural whole food ingredients together with a flavorant.

### Example 2

### Nutritional Supplement Formulation for Japanese Target Population

A nutritional supplement is formulated according to the method of the present disclosure. The target population is the Japanese people. The haplogroups for the Japanese people are identified by tracing the molecular genealogy of a representative population of the Japanese people. The most prevalent haplogroups identified in the

Japanese population are C C3, D, D2, O2, O3. The genealogical timelines of the haplogroups are traced and the relevant migratory pathways for each of the groups are identified. Based on the migratory pathways, the major geographic regions where the haplogroups spent significant time are identified. The major geographic regions which are identified are Africa, Middle East, India, Oceania, Southeast Asia, and Central Asia. The relative proportion of the haplogroups and the time spent by the haplogroups in the major geographic regions

Based on the proportion of the haplogroups in the target population and the time spent by the haplogroups in the major geographic regions the contribution of the identified major geographic regions are estimated to be: Central Asia and East Asia (taken together): about 50-55%; Middle East about 10-15%; India: about 10-15%; Oceanic: about 10-15%; Africa: 5-10%. Natural whole foods from each of the above major geographic regions are identified. Approximately 40-50 of the identified whole foods are selected for inclusion and selected based on their origin, their known cultural and historic use, and their known or postulated medicinal or therapeutic value. The amount (wt %) of each of the selected ingredients included in the formulation is approximated relative to the proportion of the haplogroups in the target population and the time spent by the haplogroups in the major geographic regions. A nutritional supplement gel is made by blending each of the selected natural whole food ingredients together with a flavorant and a gelling agent to form a nutritional supplement gel.

### Example 3

### Nutritional Supplement Formulation for Central Asian Target Population

A nutritional supplement is formulated according to the method of the present disclosure. The target population is the people of Central Asia. The haplogroups for the target population people are identified by tracing the molecular genealogy of a representative the Central Asian population. The most prevalent haplogroups identified in the Central Asian population are M89, M9, M45, LLY22, M173, M17, M20, M175, M122, M201, M174, M130, Z, D, R, A, X, C, F, and B. The genealogical timelines of the haplogroups are traced and the relevant migratory pathways for each of the groups are identified. Based on the migratory pathways, the major geographic regions where the haplogroups spent significant time are identified. The major geographic regions which are identified are Africa, Middle East, Europe, Siberia, India, Oceania, and Southeast Asia.

Based on the proportion of the haplogroups in the target population and the time spent by the haplogroups in the major geographic regions the contribution of the identified major geographic regions are estimated to be: Middle East (Region 1): about 50-55%; Europe/Siberia(taken together as Region 2): about 20-25%; India (Region 3): about 12-18%; and Oceanic/East Asia (taken together as Region 4): about 12-18%, and Africa: about 5-10%. Natural whole foods from each of the above major geographic regions are identified. Approximately 40-50 of the identified whole foods are selected for inclusion and selected based on their origin, their known cultural and historic use, and their known or postulated medicinal or therapeutic value.

The following natural whole foods are identified as possible candidates for inclusion in the nutritional supplement. The natural whole foods are listed by the geographic region to which they pertain. Region 1: marigold, carrot, flax seed, pomegranate, milk thistle, fenugreek, fig, ferula hurmonis zallouh, date, borage, parsley, humus and organic plant material that has been compressed by layers of rock, pistachio, walnut, citrus, lavender. lavandula officinalis, lemon balm, saffron, butcher's broom. Region 2: eluethero root, siberian ginseng, rhodiola, black currant, elderberry, bilberry, hawthorne berry, spinach, watercress, dandelion, rose hips, and laver/nori. Region 3: amla, olive leaf, turmeric, ashwaganda, ginger, boswellia, black pepper, and noni. Region 4: pagoda tree flower bud, polygonum cuspidatum, mangosteen, coconut, marine phytoplankton, and kelp. Region 5: devil's claw, aloe vera, chamomile, and black seed.

The amount (wt %) of each of the selected ingredients included in the formulation is approximated relative to the proportion of the haplogroups in the target population and the time spent by the haplogroups in the major geographic regions. A powdered nutritional supplement is made by blending each of the selected natural whole food ingredients together with a flavorant.

While the forgoing examples are illustrative of the principles of the present disclosure in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the invention be limited, except as by the claims set forth below.

What is claimed is:

1. A method of formulating a nutritional supplement, comprising:
a. identifying a target population;
b. identifying major haplogroups present in the target population;
c. identifying the ancestral migratory pathways for at the major haplogroups present in the target population;
d. identifying major geographic regions along each ancestral migratory pathway where the major haplogroups spent significant time;
e. selecting natural whole foods which are found indigenously in at least one of the major geographic regions; and
f. incorporating the natural whole foods into a nutritional supplement.

2. The method of claim 1, wherein the target population is people in North America.

3. The method of claim 1, wherein the target population is people of Japan.

4. The method of claim 1, wherein the target population is people in Central Asia.

5. The method of claim 1, wherein the major haplogroups are selected from the group consisting of mitochondrial haplogroups A, B, C, D, F, H, HV1, HV, I, J, K, L0, L1, L2, L3, M1, M, N1, N, Pre HV, R, T, U5, U6, U, V, W, X, Z; and Y-DNA haplogroups M119-O1a, M91-A, M60-B, M217-C3, M130-C, M15-D1, P37.1-D2, M174-D, M2-E3a, M35-E3b, M96-E, M89-F, P15-G2, M201-G, M52-H1, M69-H, M253-I1a, P37.2-I1b, M223-I1c, M170-I, M267-J1, M172-J2, M304-J, M70-K2, M9-K, M9-K*, M20-L, LLY22-N, M168, M4-M, P31-O2, M122-O3, M175-O, M45-P, M3-Q3, M242-Q, M173-R1, M17-R1a1, M343-R1b, M124-R2, M207-R, YAP; and combinations thereof.

6. The method of claim 1, wherein the major haplogroups include mitochondrial DNA haplogroups A, B, C, D, F, H, HV1, HV, I, J, K, L0, L1, L2, L3, M1, M, N1, N, R, T, U5, U6, U, V, W, X, Z; and Y-DNA haplogroups M119, M91, M217, M130, M15, P37.1, M174, M2, M35, M96, M89, P15, M201, M52, M69, M253, P37.2, M223, M170, M267, M172, M304, M170, M9, M20, LLY22, M4, P31, M122, M175, M45, M3, M242, M173, M17, M343, M124, M207.

7. The method of claim 1, wherein the major geographic regions are selected from the group consisting of Africa, Mediterranean, Middle East, Europe, Central Asia, India, Siberia, Southeast Asia, Eastern Asia, Oceania, and combinations thereof.

8. The method of claim 1, wherein the natural whole foods are selected from the group consisting of polygonum cuspidatum, amla, olive leaf, turmeric, curcumin, astragalus, fo-ti, green tea, ginkgo biloba, broccoli, pagoda tree flower bud, goji berry, mangosteen, noni, asian ginseng, eleuthero siberian ginseng, ashwaghanda, rhodiola, reishi mushroom, cordyceps, schizandra berry, chlorella, spirulina, barley grass, wheat grass, ginger, boswellia, black pepper, dandelion, grapes, grapeseed, black seed, tribulus, marigold, carrot, flax seed, pomegranate, black currant, elderberry, bilberry, hawthorne berry, holy basil, kale, brussels sprouts, spinach, broccoli sprouts, devils claw, aloe vera, chamomile, coconut, marine phytoplankton, kelp, bitter orange, sweet orange, lemon, fulvic acid/humic acid plant based trace minerals, luohanguo, honey, guggul, marine algae, cinnamon, milk thistle, fenugreek, fig, ferula hurmonis zallouh, banaba, tongkat ali, kakadu plum, date, borage, parsley, pistachio, walnut, lavandula officinalis, lemon balm, saffron, butcher's broom, watercress, rose hips, laver/nori, bladderwrack, fermented yeast/inactive nutritional yeast, cranberry, maitake, marulu tree fruit, yohimbe, roobios, lychee, arjuna, kigelia, white tea, hoodia, hemp, kava kava, epimedium sagittatum, and combinations thereof.

9. The method of claim 1, wherein the selecting step further includes selecting indigenous foods which have at least one of a) scientific evidence of their health benefits or b) known historical/cultural use.

10. The method of claim 1, wherein the nutritional supplement is formulated as powder, gel, liquid, and combinations thereof.

11. The method of claim 1, wherein the natural whole foods incorporated into the nutritional supplement are grown in the major geographic region to which they are indigenous.

12. The method of claim 1, wherein the natural whole foods are incorporated into the nutritional supplement at a relative ratio based on the haplogroups present in the target population.

13. The method of claim 1, wherein the natural whole foods are incorporated into the nutritional supplement at a relative ratio based on relative duration of time the haplogroups of the target population spent in each major geographic region.

14. A nutritional supplement formulated by the method of claim 1.

15. A nutritional supplement, comprising:
a collection of natural whole food components, said collection of natural whole food components comprising a first group of ingredients, a second group of ingredients, a third group of ingredients, and a fourth group of ingredients,
wherein said first group of ingredients comprises at least three first group ingredients selected from the group consisting of a polygonum cuspidatum, amla, turmeric, curcumin, astragalus, fo-ti, green tea, ginko biloba, broccoli, pagoda tree flower bud, goji berry, mangosteen, noni, asian ginseng, eleuthero siberian ginseng, ashwaghanda, rhodiola, reishi mushroom, cordyceps, schizandra berry, chlorella, spirulina, barley grass, wheat grass, ginger, boswellia, holy basil, bitter orange, sweet orange, lemon, luohanguo, guggul, cinnamon, maitake, lychee, arjuna, white tea, hemp, epimedium sagittatum, dandelion, chamomile, flax seed, black seed, fermented yeast/inactive nutritional yeast, and black pepper, and wherein the first group of ingredients comprises about 55 wt % to about 65 wt % of the collection of natural whole food components;
wherein said second group of ingredients comprises at least two second group ingredients selected from the group consisting of olive leaf, chamomile, dandelion, grapes, grapeseed, tribulus, marigold, carrot, flax seed, pomegranate, black currant, elderberry, bilberry, hawthorne berry, kale, brussels sprouts, spinach, fulvic acid/humic acid plant based trace minerals, honey, milk thistle, fenugreek, fig, ferula hurmonis zallouh, date, borage, parsley, pistachio, walnut, lavandula officinalis, lemon balm, saffron, butcher's broom, watercress, rose hips, and, broccoli sprouts, and wherein the second group of ingredients comprises about 25 wt % to 25 wt % of the collection of natural whole food components;
wherein said third group of ingredients comprises at least one third group ingredient selected from the group consisting of devils claw, marulu tree fruit, yohimbe, roobios, kigelia, hoodia, black seed, chamomile, and aloe vera, and wherein the third group of ingredients comprises about 5 wt % to 15 wt % of the collection of natural whole food components; and
wherein said fourth group of ingredients comprises at least one fourth group ingredient selected from the group consisting of noni, coconut, marine phytoplankton, marine algae, banaba, tongkat ali, kakadu plum, kava kava, bitter orange, laver/nori, bladderwrack, and kelp, and wherein the fourth group of ingredients comprises about 5 wt % to about 15 wt % of the collection of natural whole food components.

16. A nutritional supplement as in claim 15, wherein the nutritional supplement is formulated as a powder, gel, or liquid.

17. A nutritional supplement as in claim 15, wherein the nutritional supplement further includes a flavorant which is not an ingredient of the collection of natural whole food components.

18. A nutritional supplement as in claim 17, wherein the flavorant is a natural whole food component.

19. A nutritional supplement as in claim 15, wherein the nutritional supplement includes a gelling agent.

20. A nutritional supplement as in claim 15, wherein the first group of ingredients comprises about 57 wt % to about 63 wt % of the collection of natural whole food components.

21. A nutritional supplement as in claim 15, wherein the first group of ingredients comprises about 59 wt % to about 61 wt % of the collection of natural whole food components.

22. A nutritional supplement as in claim 15, wherein the second group of ingredients comprises about 27 wt % to about 33 wt % of the collection of natural whole food components.

23. A nutritional supplement as in claim 15, wherein the second group of ingredients comprises about 29 wt % to about 61 wt % of the collection of natural whole food components.

24. A nutritional supplement as in claim 15, wherein the third group of ingredients comprises about 7 wt % to about 13 wt % of the collection of natural whole food components.

25. A nutritional supplement as in claim 15, wherein the third group of ingredients comprises about 9 wt % to about 11 wt % of the collection of natural whole food components.

26. A nutritional supplement as in claim 15, wherein the fourth group of ingredients comprises about 7 wt % to about 13 wt % of the collection of natural whole food components.

27. A nutritional supplement as in claim 15, wherein the fourth group of ingredients comprises about 9 wt % to about 11 wt % of the collection of natural whole food components.

28. A nutritional supplement as in claim 15, wherein the collection of natural whole food components includes at least 40 different ingredients.

29. A nutritional supplement as in claim 15, wherein the first group of ingredients comprises at least 7 first group ingredients.

30. A nutritional supplement as in claim 15, wherein the second group of ingredients comprises at least 5 second group ingredients.

31. A nutritional supplement as in claim 15, wherein the third group of ingredients comprises at least 2 third group ingredients.

32. A nutritional supplement as in claim 15, wherein the fourth group of
